(19) United States
(12) Patent Application Publication
(10) Pub. No.: US 2008/0287980 A1
(43) Pub. Date: Nov. 20, 2008

(54) Divisible Cartridge for Piercing and Positioning a Body Jewellery

(75) Inventors: Karl Blomdahl, Halmstad (SE); Lars Blomdahl, Haverdal (SE)

Correspondence Address: VOLPE AND KOENIG, P.C., UNITED PLAZA, SUITE 1600, 30 SOUTH 17TH STREET, PHILADELPHIA, PA, 19103, US

(73) Assignee: BLOMDAHL MEDICAL AKTIEBOLAG, Halmstad (SE)

(21) Appl. No.: 12/094,970

(22) PCT Filed: Nov. 28, 2006
(86) PCT No.: PCT/SE2006/001357

§ 371 Date: May 23, 2008

(30) Foreign Application Priority Data
Nov. 28, 2005 (SE) 0502601-8
Nov. 28, 2005 (SE) 0502603-4

(51) Int. Cl. A44C 7/00; A61B 17/34

(52) U.S. Cl. 606/188; 606/185

(57) ABSTRACT

A cartridge for piercing in a body part for positioning a piece of body jewelry comprises a bar and a locking mechanism. The cartridge comprises two sections which are articulated to each other along a pivot and which are movable between a closed state and an open state by being pivoted on said pivot, and a locking member for locking the sections in their closed state. In their closed state, the sections define a space for receiving said piece of body jewelry, an opening and a guiding surface. The bar of the piece of body jewelry is arranged to be pushed out of the opening while the bar is being guided by said opening and while the locking means of the piece of body jewelry is being guided by said guiding surface. In their open state, the sections allow the piece of body jewelry to be released from the cartridge after completed positioning.

### TECHNICAL FIELD

The present invention relates to a cartridge for piercing in body parts for positioning a piece of body jewelry.

### TECHNICAL BACKGROUND

Piercing in body parts is frequent and performed either manually or by means of some kind of instrument. In all surgical operations, including piercing and positioning of jewelry in body parts, it is desirable to use products and methods that permit and facilitate aseptic handling. By “asepsis” is meant methods and techniques aimed at preventing the spreading of pathogenic microorganisms. Since piercing in body parts for positioning body jewelry is often performed by non-medically trained staff, it is very important that the products and methods applied allow simple aseptic handling. In recent years, it has therefore become increasingly common to use sterile disposable cartridges which contain the piece of jewelry. These cartridges are used separately or in combination with an instrument by means of which the piece of body jewelry is pushed out of the cartridge and further into and through the body part in which it is to be positioned.

To guide the piece of body jewelry in these cartridges, use is made essentially of two principles. According to the first principle, the guiding is obtained by cooperation between the cartridge and, respectively, the locking means and the bar of the piece of body jewelry. According to the second principle, the guiding is obtained by cooperation only between the cartridge and the locking means of the piece of body jewelry.

An example of the first principle is disclosed in U.S. Pat. No. 4,030,507. The cartridge comprises a holder and an elongate two-piece sleeve which is movable in the holder and in which the piece of body jewelry is contained. The cartridge is intended to be used in combination with an instrument. The two halves of the sleeve are articulated to each other by a pivot provided with an opening at the end of the sleeve that is intended to face the body part when piercing. In the piercing process, the piece of body jewelry is pressed axially through the sleeve, whereby the bar is pressed out of the opening in the pivot. At the same time, the sleeve is displaced relative to the holder. During the pushing-out movement, the bar of the piece of body jewelry is guided by the opening while its locking means is guided by the inner lateral area of the sleeve. As the locking means of the piece of body jewelry reaches the pivot, the pivot is split open, whereby the halves fall apart and uncover the piece of body jewelry. At the moment the splitting is initiated, the control over the guiding is lost, which means that there is a risk of poor guiding of the piece of body jewelry.

The second principle can be exemplified by a cartridge comprising a holder with a two-piece insert. The cartridge is used either separately or in combination with an instrument. The insert grips the locking means of the piece of body jewelry and a portion of the bar closest to the locking means. The major part of the bar is completely free. When piercing, the insert with the gripped piece of body jewelry is pressed forward a distance which is necessary for the bar to penetrate the body part involved and optionally also to engage an external locking means. After piercing, or immediately before the completion thereof, the piece of body jewelry is uncovered by the two-piece holder falling apart or being forced apart.

This principle too implies a risk of incorrect piercing or piercing with inferior quality since the bar is not guided. In addition, the fact that the bar is visible and exposed to the ambient air involves a risk of contamination of the bar before piercing, which may increase the risk of infection.

Both examples described above require a certain play between the insert and the holder to allow relative displacement between them. The play as such implies a risk of inferior guiding.

Inferior guiding involves an apparent risk that the piercing will not be performed through the intended positions of the entry and exit holes. If the piercing is not performed through the intended positions and, if the worst comes to the worst, this results in incorrect piercing which has to be done again or which causes unnecessary trauma or discomfort. Problems may also arise during the healing process in those cases where the length of the passage through the body part does not correspond to the length of the bar. Inferior guiding may also result in the bar not being correctly engaged with an external locking means, if present. If the engagement with the external locking means is incorrect or inferior, this has to be adjusted afterwards by the operator, which means discomfort for the person being pierced and, above all, a serious risk of infection for the operator as well as for the person being pierced. These risks of infection involve, among other things, microorganisms which may cause serious wound infections and highly dangerous blood viruses, such as HIV and hepatitis.

### OBJECTS OF THE PRESENT INVENTION

The object of the present invention is to provide a cartridge for piercing in body parts for positioning a piece of body jewelry, which cartridge ensures excellent guiding of the piece of body jewelry, thus permitting high quality piercing.

Another object is that the cartridge should be applicable irrespective of whether the body jewelry has a straight or bent bar.

Another object is that the cartridge should be applicable irrespective of whether it is intended to be used together with an integrated or a loose or a releasably arranged piercing means.

Another object is that the cartridge should allow aseptic handling, i.e. it should minimise the risk of infection and blood infection for both the person being pierced and receiving the piece of body jewelry and the operator.

Yet another object is that the cartridge should be applicable both separately and in the form a unit included in a system.

### SUMMARY OF THE INVENTION

To achieve at least one of the objects stated above and further objects not mentioned above, which will be apparent from the following description, the present invention relates to a cartridge for piercing and positioning a piece of body jewelry in a body part. The term “body jewelry” is meant to include ear jewelry as well as jewelry intended for other parts of the body. The piece of body jewelry comprises a locking means and a bar, which bar can be straight or bent. In the following, the term “bar” relates to a bar which is part of a body jewelry item and which is adapted to be pushed through a body part in such a manner that a through hole is made in the body part. To this end, the bar comprises a piercing means, i.e. a sharp means suitable for piercing. The piercing means can be integrated with the bar in the form of a sharp tip on its front end portion, which tip can be intended to be removed or not removed after piercing. The piercing means can also be arranged on the front end portion of the bar or releasably arranged in connection therewith, for instance in the form a cannula.

In connection with body jewelry, the term “locking means” relates to a means which is arranged on the bar and which prevents the bar from being withdrawn from the body part. At the same time, the locking means can be a piece of jewelry. It can be integrated with the bar or constitute a separate unit, which is adapted to be removable from and applicable to the bar.

The term “body part” is meant to also include mucous membrane.

According to a first aspect, the invention relates to a cartridge for piercing and positioning a piece of body jewelry comprising a bar and a locking means in a body part, which cartridge comprises two sections which are articulated to each other along a pivot and which are movable between a closed state and an open state by being pivoted on said pivot, and a locking member for locking the sections in their closed state, which sections, in their closed state, define a space for receiving said piece of body jewelry, an opening and a guiding surface, the bar of the piece of body jewelry being arranged to be pushed out of the opening while the bar is being guided by said opening and while the locking means of the piece of body jewelry is being guided by said guiding surface, and the sections allowing, in their open state, the piece of body jewelry to be released from the cartridge after completed positioning.

In this way, a cartridge is obtained which, thanks to the space formed, can contain and protect a piece of body jewelry in a sterile manner until it is to be used.

The piece of body jewelry is kept in place in a stable manner in the space of the cartridge by means of at least the guiding surface, from the moment the piece of body jewelry is mounted in the cartridge until the cartridge is used, which is an important aspect to ensure correct piercing and positioning of a piece of body jewelry. Furthermore, the piece of body jewelry obtains stable guiding during piercing and positioning since the bar, while being pushed out, is guided by the opening while the locking means is guided by the guiding surface. The stability is maintained, also after the bar has been pushed out of the opening, the distance required for the bar to penetrate the body part involved and possibly also lockingly engage an external locking means. This effect is achieved due to the fact that the sections are moved to their open state only after completed piercing to allow the piece of body jewelry to be released from the cartridge. Until then, the contact is maintained between the locking means and the guiding surface and between the bar and the opening. The operator is thus free to decide when he is ready to open the cartridge and release the control over the piece of body jewelry. The operator may, for instance, wish to perform a visual inspection of the final result, remove an optional loosely arranged piercing means or mount an external locking means. In the case of an inexperienced operator, it may also be an advantage to have free control over the opening of the cartridge since then the stress and thus also the risk of mistakes can be reduced.

The guiding provided by the two sections through the combination of the guiding of the bar by the opening and the guiding of the locking means by the guiding surface results in very high quality piercing since the piercing and the positioning can be performed through the desired positions of the entry hole and the exit hole, or at least through the position of the desired entry hole. It will be understood that this is the case provided that the cartridge is correctly positioned relative to these positions before and during piercing. Furthermore, it is possible to obtain a correct engagement with an external locking means, if present. This means that the entire operation including piercing, positioning of the piece of body jewelry and application of an external locking means can be performed in sequence, without unnecessary contact between the operator and the body part, which reduces the risk of infection.

The cartridge is applicable, irrespective of the geometry of the bar and the locking means, i.e. whether the bar is straight or bent or whether the locking means is rotationally symmetrical in cross-section or not.

A pressing means can be movable in the space to push at least part of the bar out of the opening. The pressing means can be integrated into the cartridge or, alternatively, arranged as a unit which is separate from the cartridge and with which the cartridge is intended to cooperate during piercing and positioning.

In a first embodiment, the locking member can comprise a snap lock arrangement which moves into engagement when the sections are brought together to be locked in their closed state. An actuating means can be arranged for disengaging the snap lock arrangement to allow the sections to be moved to their open state. The actuating means can, for instance, be adapted to disengage the snap lock arrangement by wedging action, which urges the sections to their open state. The actuating means can thus be operated manually by means of a pulling or pushing motion, depending on the locking direction of the wedge. It may also be actuatable by the pressing means, i.e. so that the pressing means, when displaced relative to the cartridge, contacts the actuating means. As an alternative, the pressing means can be adapted to engage the actuating means after completed positioning of the piece of body jewelry. In the latter case, the required movement of the pressing means and the actuating means, respectively, can be adapted so as to correspond to a movement of the bar into the position in which the bar is pushed out of the opening of the cartridge a distance such that it has not only penetrated the body part but also lockingly engaged an external locking means.

In another embodiment, the locking member can comprise a shaft provided with at least one projection, which shaft is supported by one of the at least two sections, and which projection, when the shaft is displaced relative to the sections, is arranged to lockingly engage at least one space formed in the other of the at least two sections. The shaft formed with at least one projection can be movable to disengage from said space by cooperation between the pressing means and the shaft provided with at least one projection. Furthermore, the shaft provided with at least one projection can be movable to disengage from said space by cooperation between the pressing means and the shaft provided with at least one projection after completed positioning. In the latter case, the required movement of the pressing means and the actuating means, respectively, can be adapted so as to correspond to a movement of the bar into the position in which the bar is pushed out of the opening of the cartridge a distance such that it has not only penetrated the body part but also lockingly engaged an external locking means.

According to another alternative embodiment, said locking member can comprise a cotter pin, which is arranged to lockingly engage the sections in their closed state. The cotter pin can easily be pulled out after completed positioning, whereby the sections can be moved to their open state to allow the release of the parts of the piece of body jewelry that are still contained in the cartridge after completed positioning.

In still another alternative embodiment, the locking member can be a unit separate from the sections. Such a unit can, for instance, be a clip or sleeve which is arranged to grasp over the sections to lock them in their closed state. The locking member can also be integrated with at least one of the sections. The term “integrated” here means that the parts of the locking member are attached in some way to the sections so that the locking member can be operated for locking or unlocking. This can be achieved, for instance, by these parts of the locking member being injection moulded in one piece with the sections or fastened thereto in some other way.

According to an alternative embodiment, the at least two sections can be articulated to each other by cooperation with a unit separate from the cartridge. Such a unit can, for instance, be a holder, an instrument with a pressing means adapted to cooperate with the cartridge to push out a piece of body jewelry arranged therein or some kind of pinching and sighting device which is used, among other things, to grip the body part involved during positioning. Such units together form a system.

In their closed state, the two sections can define a parting line which extends substantially perpendicularly to a body part intended to be pierced. This allows the sections to be moved to their open state without being impeded by the body part, at the same time as the piece of body jewelry can be released after completed positioning.

Furthermore, the cartridge allows the use of a piercing means which is loosely or releasably connected to the bar. When using such a piercing means, a support means arranged in the cartridge can further improve the guiding of the piercing means, from the moment the piece of body jewelry is mounted with the piercing means in the cartridge and until the piece of body jewelry is positioned in a body part. This ensures high quality during piercing and positioning, but also that the rejection rate due to careless handling of the cartridge will be low. The support means can be designed in a number of ways. The space arranged in the cartridge can comprise a support means in the form of an elastic portion, which is convex relative to the internal boundary surface of the space and which is adapted to guide a piercing means which is loosely or releasably connected to the bar. Such an elastic portion can, for instance, be a thin elastic diaphragm or yielding projections or fingers, which can all be formed in connection with the injection moulding of the sections of the cartridge. The support means can also be a separate means arranged internally in the space.

The space can comprise a first groove for receiving and guiding the bar and a second groove forming said guiding surface for receiving and guiding the locking means, the first and second grooves merging adjacent to the opening. This solution is advantageous if the piece of body jewelry has a bent bar. In this case, the bar can be guided, when it is pushed out, by the opening as well as by said first groove.

The cartridge can be arranged to cooperate with a pinching and sighting device, which is adapted to grip and to take aim at least one entry hole intended for piercing in a body part which is to be pierced and to allow orientation of the opening formed in the cartridge relative to said entry hole.

By the cooperation of the cartridge with a pinching and sighting device, the quality of the piercing can be increased, of course, provided that the body part has been correctly positioned and gripped by the pinching and sighting device in such a manner that at least the desired entry hole is arranged in alignment with the opening of the cartridge. Optimal alignment is achieved if the positions of both the desired entry hole and the desired exit hole are oriented opposite to each other when the body part is gripped by the pinching and sighting device. If this is the case, the cartridge, by its arrangement on the pinching and sighting device, will ensure that the piercing is performed through the positions of these holes. If the cartridge is arranged on the pinching and sighting device only after the desired positions of the entry hole and the exit hole, or at least the position of the desired entry hole, have been aligned and gripped by the pinching and sighting device, a clear division is obtained between the sighting stage and the piercing stage, which allows the entire piercing operation to be more relaxed, thus minimising the risk of incorrect piercing.

The cartridge can also comprise or be arranged to cooperate with an abutment, such that the sections and the abutment are stationary arranged when the bar is pushed out. This results in very stable guiding of the piece of body jewelry during the positioning thereof in a body part.

### DESCRIPTION OF THE DRAWINGS

The invention will now be described in more detail for the purpose of exemplification with reference to the accompanying drawings, which show a currently preferred embodiment of the device.

FIGS. 1 and 2 are perspective views, seen obliquely from, respectively, the front and the rear, of an embodiment of the cartridge.

FIG. 3 shows a section of the cartridge intended for a piece of body jewelry with a straight bar.

FIG. 4 shows an embodiment of a section of the cartridge intended for a piece of body jewelry with a bent bar.

FIG. 5 shows the section according to FIG. 4 in a position after positioning when the cartridge is ready to be opened.

FIG. 6 shows an embodiment of a locking member.

FIGS. 7a and 7b are schematic views of the locking member shown in FIG. 6, showing the engagement of the locking shaft and the sections.

FIG. 8 shows yet another embodiment of a locking member.

FIG. 9 shows a cartridge arranged on a pinching and sighting device.

FIGS. 10a and 10b schematically show the cartridge with an integrated abutment and in cooperation with a separate abutment, respectively.

FIG. 11 schematically shows an embodiment of a locking member separate from the sections.

### TECHNICAL DESCRIPTION

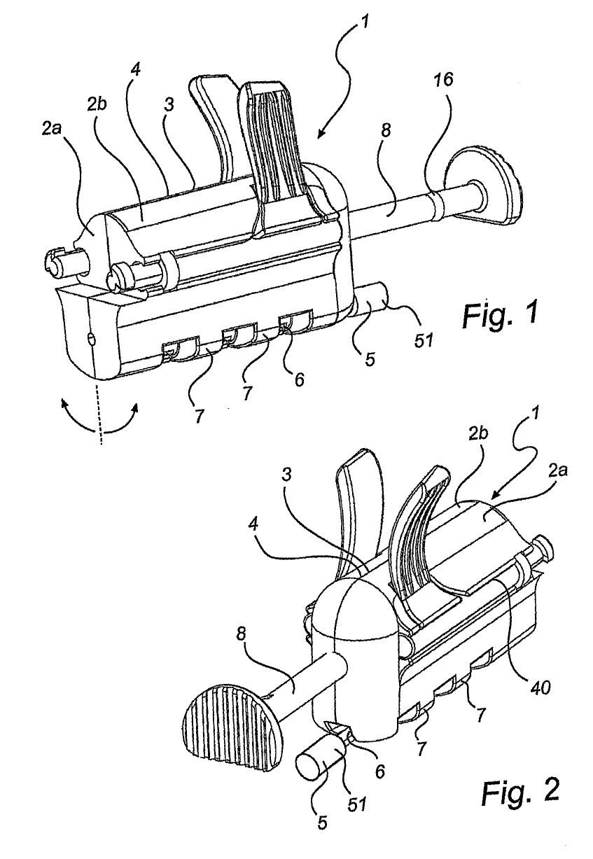

With reference to FIGS. 1 and 2, an embodiment of the inventive cartridge 1 is shown obliquely from the front and obliquely from the rear, respectively.

The cartridge 1 comprises two sections 2a, 2b, which are articulated to each other by a pivot 3. In FIG. 1, arrows schematically show how the sections 2a, 2b are adapted to be moved apart. In the shown embodiment, the pivot 3 is arranged on top of the cartridge. It will be understood that the pivot 3 can be arranged with the same function in some other position on the cartridge, provided that the sections 2a, 2b can be moved to their open state to allow release of a piece of body jewelry from the cartridge 1 after completed positioning. The pivot 3 should also be located such that the sections 2a, 2b, in their closed state, define a parting line 4 which extends substantially perpendicularly to the body part in which the piercing and positioning is to be performed.

In the shown embodiment, the pivot 3 is formed of locally thinner material, which means that the two sections are injection moulded in one piece. The pivot 3 can also be obtained in some other way, for instance, by complementary hinge means (not shown) arranged on the respective sections, whereby the two sections are snapped together when mounted to form a pivot. The pivot can also be created by the cooperation between the sections and a separate unit.

As stated above, the cartridge here comprises two sections 2a, 2b, but it will be understood that it can be formed in more than two sections.

In the shown embodiment, the two sections 2a, 2b are interlockable on the underside of the cartridge 1 to define a space in the form of a closed cavity. In the shown embodiment, this is achieved by means of a locking member 5 comprising a withdrawable cotter pin 51 which is arranged through a channel 6 defined by fingers 7 on the underside of the respective sections 2a, 2b. The fingers 7 are mutually arranged so as to mesh with each other and define a channel 6 in the closed state of the two sections 2a, 2b. It will be understood that the shown embodiment of the locking member is only one possible embodiment, two other embodiments being described below.

In the shown embodiment, the cartridge also comprises a pressing means 8 which is movable inside the cartridge 1 to push out at least part of the bar of a piece of body jewelry arranged in the same. The pressing means 8 can have a stop means 16 at its rear end adapted to engage the cartridge 1 when the pressing means is in its pushed-in position. Owing to this, the pressing means 8 can function as an abutment when an external locking means is to be mounted on a piece of body jewelry already positioned.

The pressing means 8 can either, as shown, be movably arranged in the cartridge 1 as a part thereof or be included in some other unit. As an example of such other unit, mention can be made of an instrument, for instance of multiple-use type, by means of which the cartridge and/or some form of pinching and sighting device can be operated, or a unit by means of which the cartridge can be arranged on a pinching and sighting device.

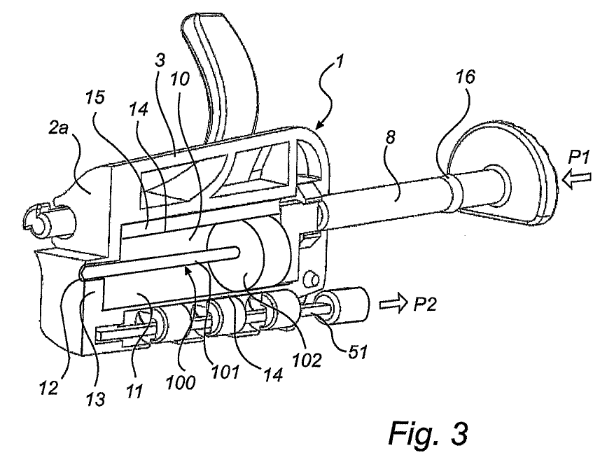

With reference to FIG. 3, the interior of the cartridge 1 will now be described with reference to a cartridge which is intended for a piece of body jewelry with a straight bar. In case of two sections, it is advantageous if they mirror each other, and therefore only one section 2a will be described. The section 2a comprises an internal boundary surface 10. When bringing two sections together, their respective boundary surfaces together define a space 11 which is adapted to contain a piece of body jewelry 100 comprising a bar 101 with a locking means 102 arranged on its rear end portion. The locking means 102 can, for instance, be a piece of jewelry.

The boundary surface 10 defines an opening 12 at the front end of the cartridge 1, i.e. the end intended to be facing the body part in which the piece of body jewelry is to be positioned, by which opening the space 11 communicates with the ambient air and through which at least part of the bar 101 can be pushed out by the pressing means 8 described above. The opening 12, which is formed by the material 13 of the lateral area of the opening, forms a passage which allows the bar 101 of the piece of body jewelry 100 to be guided when pushed out of the cartridge 1. In the shown embodiment, the opening 12 is circular but it can have a number of different cross-sections as long as it ensures guiding of the bar 101 in the axial direction and in the lateral direction.

The boundary surface 10 further forms a guiding surface 14 which is adapted to guide the locking means 102 when the bar 101 is pushed out of the opening 12. The opening 12 and the guiding surface 14 are axially aligned with each other and have such cross-sectional geometry that they ensure stable guiding of the piece of body jewelry 100 in the axial direction and in the lateral direction when the bar 101 is pushed out. Advantageously, the guiding surface 14 and the opening 12, respectively, surround the locking means and the bar, respectively, in the circumferential direction but can also be divided in sections. The guiding surface 14 advantageously has an extension in the axial direction so as to provide guiding during the entire pushing-out operation. It will be understood that the locking means 102 can have any geometry and still provide satisfactory guiding as long as the guiding surface is adapted to this geometry.

The boundary surface 10 further comprises a recess 15 for receiving and guiding the above-mentioned pressing means 8. The recess 15 extends in the working direction of the pressing means 8 and is adapted to guide the same in the axial direction but also in the lateral direction. The recess 15 can be arranged in only one of the sections 2a, 2b or be arranged by cooperation between the two sections.

When a pressure P1 is applied to the pressing means 8, the pressing means 8 advances towards the opening 12 to push part of the bar 101 out of the opening. In this motion, the piece of body jewelry 100 will be displaced in the space 11 at the same time as the bar 101 is guided by the opening 12 and the locking means 102 is guided by the guiding surface 14. When the bar 101 has been pushed out of the opening 12 a distance long enough to have penetrated the body part involved and possibly also lockingly engaged an external locking means, the piece of body jewelry 100, with its locking means 102 and the part of the bar 101 that is still in the opening 12, is still contained in the cartridge 1 and in contact with the opening 12 and the guiding surface 14, respectively. Although the positioning is now completed, the piece of body jewelry is thus still contained in the cartridge such that the operator has full control over the piece of body jewelry.

To uncover the piece of body jewelry 100 after completed positioning, the cartridge 1 thus has to be opened, which is performed by the two sections 2a, 2b being moved apart. In the shown embodiment, this is obtained by the operator withdrawing the above-mentioned cotter pin 51, by applying a force P2, whereby the two sections 2a, 2b can be pivoted apart by a rotary motion on said pivot 3. The rotary motion is schematically shown by arrows in FIG. 1. Since the two sections 2a, 2b, in their closed state, define a parting line 4 which extends substantially perpendicularly to a body part which is to be pierced, the sections can be moved apart without being impeded by the body part. The cartridge can thus be removed from the piece of body jewelry and the body part without any unnecessary contact therewith.

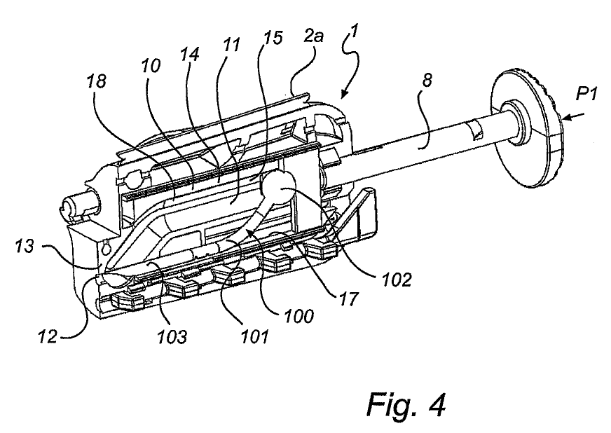

With reference to FIG. 4, an interior is shown of a section 2a of a cartridge 1 which is intended for a piece of body jewelry with a bent bar. The section 2a is shown with a piece of body jewelry 100 and a piercing means 103 which is loosely arranged on the bar 101. The piece of body jewelry 100 and the piercing means 101 are contained in the space 11 defined between two closed sections 2a, 2b, of which only one is shown.

Like in the above-described cartridge, which is intended for a piece of body jewelry with a straight bar, the boundary surface 10, at the front end of the cartridge, defines an opening 12 by which the space 11 can communicate with the ambient air and through which at least part of the bar 101 can be pushed out. The opening 12 formed by the material 13 of the lateral surface of the opening creates a passage which provides guiding of the bar 101 of the piece of body jewelry 100 and the piercing means 103 loosely arranged thereto when the bar is pushed out of the cartridge. In the shown embodiment, the opening 12 is circular but it may have a number of different cross-sections as long as it ensures guiding of the bar and the piercing means in the axial direction and in the lateral direction when they are pushed out of the opening.

Furthermore, the boundary surface 10 defines, inside the cartridge 1, a lower 17 and an upper 18 groove. In the shown embodiment, the upper groove 18 is oriented towards the lower groove 17. The piece of body jewelry 100 is arranged in the cartridge 1 in such manner that, on the one hand, the piece of jewelry is contained in a recess 15 which is formed to receive the pressing means 8 and, on the other hand, the piercing means 103 and the bar 101 are received in the lower groove 17 while the locking means 102 is received in the upper groove 18. The upper groove 18 thus forms a guiding surface 14 for the locking means 102 whereas the lower groove 17 forms a complementary guiding surface for the bar 101 and the piercing means 103.

Thanks to the fact that the upper groove 18 is oriented towards the lower groove 17, the bar, in spite of its bent shape, can be made to follow, in its passage through a body part, substantially the passage created by the piercing means through the body part when the bar is pushed out of the opening by means of a pressing means. Owing to this, trauma is minimised.

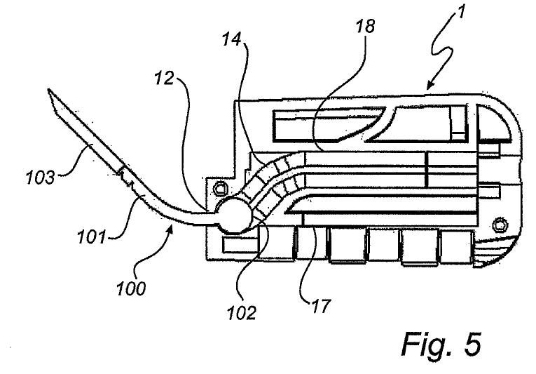

Like in the cartridge intended for a piece of body jewelry with a straight bar, the boundary surface 10 comprises a recess 15 for receiving and guiding a pressing means 8. This recess 15 has the same design as in the embodiment described above and is therefore not described again. By a pressure P1 being applied to it, the pressing means 8 advances through the cartridge 1 and by abutment against the locking means 102 the pressing means can press the piercing means 103 and part of the bar 101 out of the opening 12. The pushing-out motion can continue until the locking means 102 abuts against the material 13 adjacent to the opening 12, see FIG. 5, which for the sake of clarity shows the section without pressing means. The bar 101, and its associated piercing means 103, is now pushed out in such manner that it has a free and uncovered end portion, on which an external locking means (not shown) can be fastened. With reference to the description above of a cartridge with a straight bar, the piece of body jewelry in the inventive cartridge, with its locking means 102 and the part of the bar 101 still being located in the opening 12, is still contained in the cartridge 1 and in contact with the opening 12 and the guiding surface 14, and the operator is free to decide when he is ready to open the cartridge. The opening of the cartridge and the release of the piece of body jewelry is performed in accordance with that stated above in connection with a cartridge intended for body jewelry with a straight bar, and therefore this is not described again.

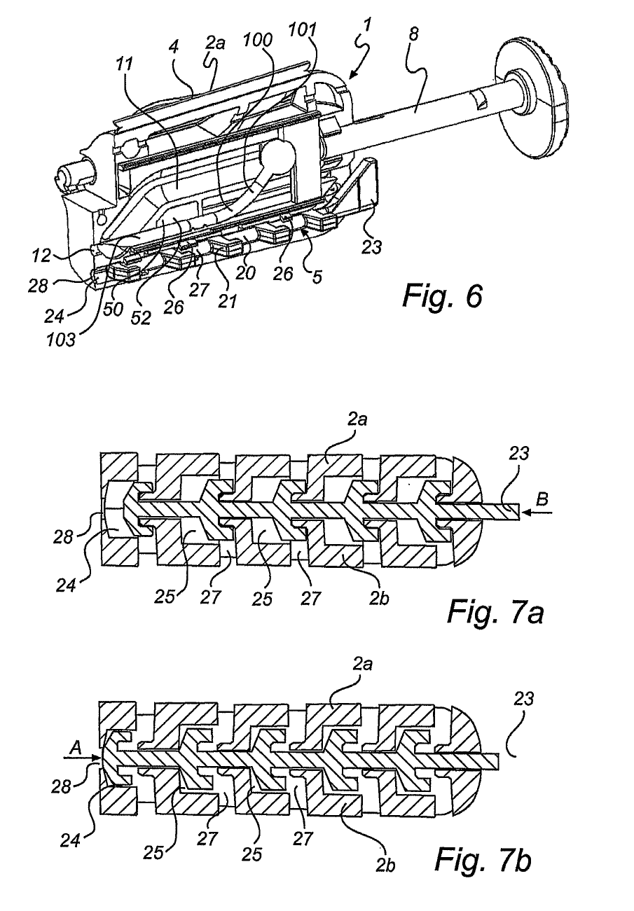

With reference to FIGS. 6 and 7, another embodiment of the locking member 5 is shown. It comprises a shaft 20 provided with hook-shaped projections 21 and an actuating means 23. In the shown embodiment, the hook-shaped projections 21 are oriented perpendicularly away from the parting line 4 of the section 2a, 2b but it will be understood that they can also be oriented in some other direction. Furthermore, the hook-shaped projections 21 are oriented such that the open hook portion 22 of the respective hook-shaped projections 21 is oriented away from the opening 12 of the cartridge. The shaft 20 is movably arranged in a groove 24 which is formed in the lower portion of one of the sections 2a between a locking position (see FIG. 7a) and an opening position (see FIG. 7b).

The locking member further comprises (see FIGS. 6, 7a and 7b) a portion with intermittently arranged recesses 25 which are arranged in both the sections 2a, 2b. The recesses 25 are separated from each other by bridges 26 which are arranged so as to create a space 27 under each bridge 26, which space the hook-shaped projections 21 of the shaft 20 can engage. The recesses 25 are arranged so that the hook-shaped projections 21 of the shaft 20 can be received in the recesses 25 when the two sections 2a, 2b are brought together and the shaft 20 is in its opening position (see FIG. 7b). In this position, the shaft 20 can be moved to its locking position (see FIG. 7a) by being displaced so that the hook-shaped projections 21 mesh with the spaces 27. In this position, the sections 2a, 2b are prevented from being separated.

The shaft 20, the recesses 25 and the spaces 27 are suitably arranged so that the shaft has to be moved backwards (arrow A), i.e. away from the opening 12 of the cartridge, to be moved from its opening position to its locking position. To make this motion possible, the cartridge suitably comprises, at its front end, a through hole 28, by which the groove 24, in which the shaft 20 runs, communicates with the ambient air. This makes it easy to actuate the locking member 5 when the piece of body jewelry 100 is mounted in the cartridge 1. After the mounting of the piece of body jewelry 100 in the cartridge 1, the two sections 2a, 2b are brought together, which causes the hook-shaped projections 21 of the shaft 20 to be received into the recesses 25. After this, a pointed object is inserted through said hole to apply a light pressure (arrow A) to the shaft 20. This pressure moves the shaft to its locking position (FIG. 7a). The spaces 27 and the hook-shaped projections 21 on the shaft 20 can be formed so as to produce a snap lock function in the axial direction.

To open the cartridge 1, a pressure is applied (arrow B) to the actuating means 23 in the direction of the opening 12 of the cartridge. Owing to this, the shaft 20 is displaced in the opposite direction, which causes the hook-shaped projections 21 to be released from the spaces 27 and placed again in the recesses 25 (see FIG. 7b), the two sections thus being free to be moved apart to allow the release of the piece of body jewelry.

The actuating means 23 can be arranged so as to cooperate with the pressing means 8, in which case the pressing means engages the actuating means when the pressing means has been pushed into the cartridge 1 a distance long enough to complete the positioning. The cooperation between the pressing means 8 and the actuating means 23 can also be arranged such that the shaft is disengaged from the recesses only after completed positioning, that is the pressing means can have a first and a second end position: a first end position when the positioning is completed and a second end position when the pressing means is displaced somewhat more after the positioning to engage the actuating means for opening of the cartridge.

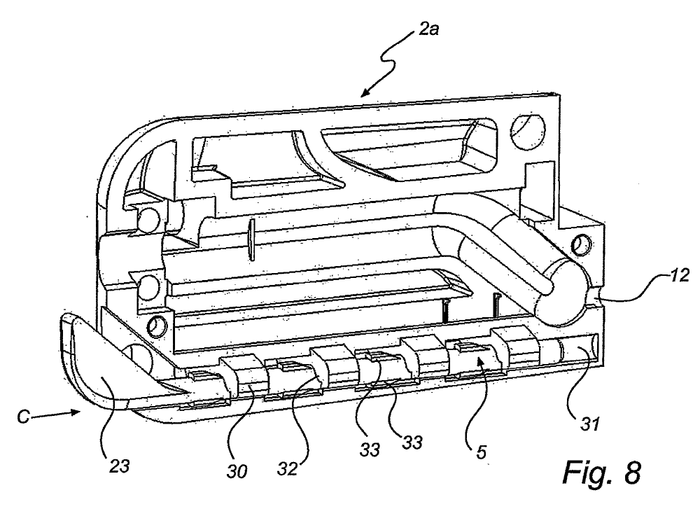

With reference to FIG. 8, yet another variant of a locking member 5 is shown. The locking member 5 is based on a combination of snap locking and wedging action. A first section 2a comprises, at its lower edge, parts of the locking member 5 in the form of hook-shaped projections 30 which are directed so that they can be pushed into locking engagement with complementary lugs (not shown) arranged in the second section (not shown). The hook-shaped projections 30 have such a profile that a channel 31 forms along the longitudinal axis of the cartridge when the two sections are brought together. This channel 31 is adapted to receive another part of the locking member 5 in the form of a shaft 32 which is provided with wedge-shaped projections 33 along its longitudinal axis. By applying a pressure (arrow C) to the shaft 32, the shaft is displaced in the channel 31 at the same time as the wedge-shaped projections 33 force apart, by wedging action, the hook-shaped projections 30 and the complementary lugs (not shown). The shaft 32 is preferably adapted to be actuated in the direction towards the opening 12 of the cartridge to make the cartridge openable. Like in the embodiment described above, the shaft 32 can comprise an actuating means 23 which can cooperate with a pressing means which, for the sake of clarity, is omitted in FIG. 8, so that the pressing means engages the actuating means 23 of the shaft when the pressing means has been pushed into the cartridge a distance long enough to complete the positioning. The cooperation between the pressing means and the actuating means can also be arranged such that the shaft is disengaged from the recesses only after completed positioning, that is the pressing means can have a first and a second end position: a first end position when the positioning is completed and a second end position when the pressing means is displaced somewhat more after the positioning to engage the actuating means for opening of the cartridge.

The above-described embodiments of the locking member can be considered to be integrated with the sections of the cartridge. The locking member can also be a unit that is separate from the sections of the cartridge. Such a locking member can be designed in a number of ways, for instance, as a sleeve or a clip grasping over the sections to lock them in their closed states. This is schematically shown in FIG. 11.

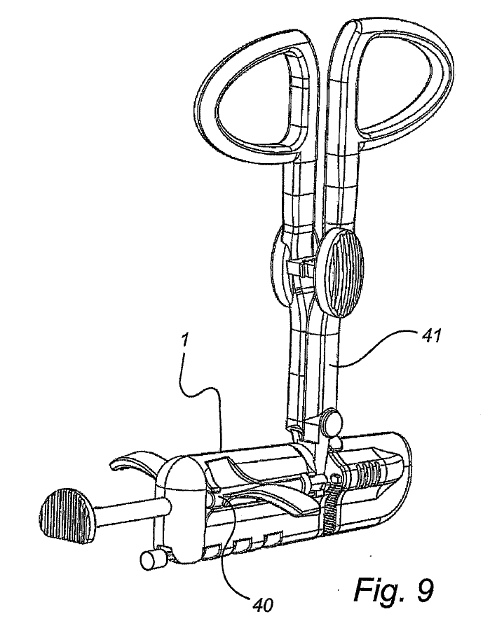

The inventive cartridge can, as described above, be used separately for piercing and positioning of a piece of body jewelry. However, it can also be used advantageously as a unit included in a system. To cooperate with other units of this kind, the cartridge can have coupling means. An example of such a coupling means 40 for cooperation with a unit in the form of a pinching and sighting device is shown in FIG. 9. The cartridge 1 is shown when mounted on a pinching and sighting device 41 which is adapted to grip a body part intended to be pierced and to allow orientation of the opening arranged in the cartridge relative to at least one entry hole intended for piercing in the body part.

In those cases where the cartridge is intended to contain a piercing means that is loosely or releasably arranged to the bar, a support means may be needed to keep the piercing means in place inside the cartridge. The support means can have a number of designs, of which a few will be described below with reference to FIG. 6.

In a first embodiment, the space 11 defined by the two sections 2a, 2b can comprise support means 50 in the form of an elastic portion 52 which is convex relative to the internal boundary surface of the space. Such an elastic portion 52 ensures stable positioning, but also guiding, of the piercing means 103 from the moment the piece of body jewelry 100 is mounted in the cartridge and until the piercing means 103 has been pushed out of the opening 12. The elastic portion 52 can, for instance, be a thin elastic diaphragm or yielding projections or fingers, which can all be formed in connection with the injection moulding of the sections of the cartridge. In FIG. 6, the portion is a diaphragm.

According to another embodiment (not shown), the support means can be a loose member which is arranged inside the space when the piece of body jewelry and the piercing means are mounted in the cartridge. Such a loose member can be displaceable inside the space in connection with the pushing-out of the bar or be fixedly attached by being squeezed between the boundary surfaces of the space.

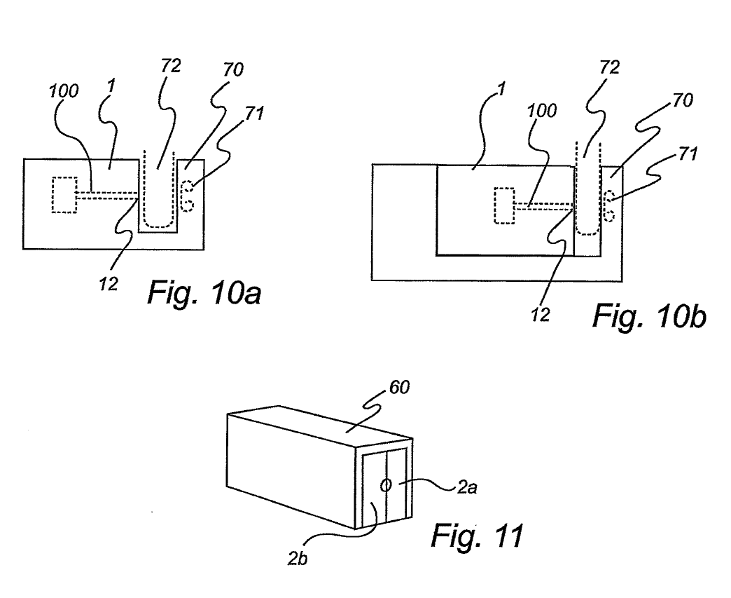

With reference to FIG. 10a, an embodiment is schematically shown in which the cartridge 1 comprises an abutment 70. The abutment 70 is arranged opposite the opening 12 of the cartridge in such manner that the body part 72 in which the piece of body jewelry 100 is to be positioned rests against the abutment and the opening, respectively. As shown, the abutment 70 can contain an external locking means 71. The external locking means can also be contained in an associated unit (not shown) in the form of a locking means holder. Furthermore, the abutment can be arranged to catch a piercing means detachably arranged to the bar.

In FIG. 10b, another embodiment is shown in which the abutment 70 is arranged as a unit detachably arranged to the cartridge 1. The abutment is arranged opposite the opening 12 of the cartridge in such manner that the body part 72 in which the piece of body jewelry 100 is to be positioned rests against the abutment and the opening, respectively.

Independently of its design, the abutment is advantageously arranged such that the sections are stationary relative to the abutment when the bar is pushed out of said opening.

It will be understood that if the cartridge is arranged to a pinching and sighting device, as shown with reference to FIG. 9, this device functions as an abutment.

It will be understood that the opening formed in the cartridge can be created when a piercing means is pressed through a diaphragm forming a front wall in the cartridge.

The cartridge and its constituent parts are preferably made by injection moulding of a polymer material.

The cartridge has been described as comprising a closed space 11, i.e. a cavity, but it can also, with the same function, have open portions (not shown) through the sections.

The cartridge is preferably provided as a sterile unit, containing a piece of body jewelry or ear jewelry and an optional associated piercing means, an optional abutment and an optional external locking means.

It will be understood that the present invention is not limited to the shown embodiments and that several variants and modifications are feasible. The invention is thus defined only by the appended claims.

1. A cartridge (1) for piercing in a body part for positioning a piece of body jewelry (100) comprising a bar (101) and a locking mechanism (102), which cartridge comprises two sections (2a, 2b) which are articulated to each other along a pivot (3) and which are movable between a closed state and an open state by being pivoted on said pivot, and a locking member (5) integrated with the sections for locking the sections in their closed state,
which sections, in their closed state, define a space (11) for receiving said piece of body jewelry, an opening (12) and a guiding surface (14),
the bar (101) of the piece of body jewelry (100) being arranged to be pushed out of the opening (12) while the bar is being guided by said opening and while the locking means (102) of the piece of body jewelry is being guided by said guiding surface (14), and
the sections (2a, 2b) allowing, in their open state, the piece of body jewelry to be released from the cartridge after completed positioning.

2. A cartridge as claimed in claim 1, in which a pressing mechanism (8) is movable in the space (11) to push at least part of the bar (101) out of the opening (12).

3. A cartridge as claimed in claim 2, in which the pressing mechanism (8) is integrated into the cartridge or, alternatively, arranged as a unit separate from the cartridge.

4. A cartridge as claimed in claim 1, in which said locking member (5) comprises a snap lock arrangement (30, 33) which moves into engagement when the sections (2a, 2b) are brought together to be locked in their closed state.

5. A cartridge as claimed in claim 4, further comprising an actuating mechanism (23) for disengaging the snap lock arrangement (30, 33) to allow the sections (2a, 2b) to be moved to their open state.

6. A cartridge as claimed in claim 5, in which the actuating mechanism (23) is adapted to disengage the snap lock arrangement (30, 33) by wedging action, which urges the sections (2a, 2b) to their open state.

7. A cartridge as claimed in claim 6, in which the actuating mechanism (23) is actuatable by the pressing means (8).

8. A cartridge as claimed in claim 7, in which the pressing mechanism (8) is adapted to engage the actuating mechanism (23) after completed positioning.

9. A cartridge as claimed in claim 1, in which said locking member (5) comprises a shaft (20) provided with at least one projection (21), which shaft is supported by one of the at least two sections (2a, 2b), and which projection, when displacing the shaft relative to the sections, is arranged to lockingly engage at least one space (27) formed in the other of the at least two sections.

10. A cartridge as claimed in claim 9, in which the shaft (20) provided with at least one projection is movable to disengage from said space (27) by cooperation between the pressing mechanism (8) and the shaft (20) provided with at least one projection.

11. A cartridge as claimed in claim 10, in which the shaft (20) provided with at least one projection is movable to disengage from said space (27) by cooperation between the pressing mechanism (8) and the shaft (20) provided with at least one projection after completed positioning.

12. A cartridge as claimed in claim 1, in which said locking member (5) comprises a cotter pin (51), which is arranged to lockingly engage the sections (2a, 2b) in their closed state.

13. A cartridge as claimed in claim 1, in which the locking member (5) is integrated with at least one of said sections (2a, 2b).

14. A cartridge as claimed in claim 1, in which said locking member (5) is a unit separate from said sections (2a, 2b).

15. A cartridge as claimed in claim 1, in which the two sections (2a, 2b), in their closed state, define a parting line (4) which extends substantially perpendicularly to a body part intended to be pierced.

16. A cartridge as claimed in claim 1, in which the space (11) comprises a support mechanism (50) for guiding a piercing mechanism (103) which is loosely or releasably connected to the bar (101).

17. A cartridge as claimed in claim 16, in which the support mechanism (50) comprises an elastic portion which is convex relative to the internal boundary surface (10) of the space.

18. A cartridge as claimed in claim 1, in which the space (11) comprises a first groove (17) for receiving and guiding the bar (101) and a second groove (18) forming said guiding surface (14) for receiving and guiding the locking means (102), the first and second grooves merging adjacent to the opening (12).

19. A cartridge as claimed in claim 1, which is arranged to cooperate with a pinching and sighting device (41), which is adapted to grip and to take aim at least one entry hole intended for piercing in a body part which is to be pierced and to allow orientation of the opening formed in the cartridge relative to said entry hole.

20. A cartridge as claimed in claim 1, further comprising an abutment (70), which is arranged such that the sections (2a, 2b) and the abutment are stationary when said bar is pushed out.

21. A cartridge as claimed in claim 1, which is arranged to cooperate with an abutment (70), which cooperation is arranged in such manner that the sections (2a, 2b) and the abutment are stationary when said bar is pushed out.
